(19) United States
(12) Patent Application Publication
(10) Pub. No.: US 2026/0000856 A1
(43) Pub. Date: Jan. 1, 2026

(54) HOLISTIC AND THERAPEUTIC BED ENCLOSURE ASSEMBLY AND METHODS OF MAKING THE SAME

(71) Applicant: Line29, Inc., San Diego, CA (US)

(72) Inventor: Casey Browar, San Diego, CA (US)

(21) Appl. No.: 19/294,166

(22) Filed: Aug. 7, 2025

Related U.S. Application Data
(63) Continuation-in-part of application No. 18/915,026, filed on Oct. 14, 2024.
(60) Provisional application No. 63/665,094, filed on Jun. 27, 2024.

(51) Int. Cl. A61M 21/00; A61M 21/02; A61N 5/06

(52) U.S. Cl. CPC A61M 21/0094; A61M 21/02; A61M 2021/0027; A61N 2005/0663; A61N 5/06

(57) ABSTRACT

A bed enclosure assembly is provided. In preferred embodiments, the bed enclosure assembly comprises a plurality of vertical walls spaced apart to form a plurality of openings coupled together by a ceiling and a floor. A plurality of doors are configured to close off the openings such that the enclosure can be completely closed off. A bed is positioned within the enclosure. The enclosure has copper cladding that covers each vertical wall in the plurality of vertical walls in its entirety. A plurality of crystals are located within the plurality of vertical walls of the bed enclosure.

### FIELD

The present patent document relates to furniture. In particular, the present patent document relates to furniture that can provide therapeutic benefits and methods of making the same. Even more specifically, the present patent application relates to the design of a therapeutic bed enclosure assembly and methods of making the same.

### BACKGROUND

In today's fast-paced society, where stress and imbalance are prevalent, many individuals are turning to alternative methods to promote holistic wellness and harmony. Holistic practices, rooted in the interconnectedness of the mind, body, and spirit, offer avenues for achieving well-being beyond conventional approaches. Central to these practices is the utilization of materials that provide unique energetic properties believed to aid in restoring balance and vitality.

Holistic wellness embraces the integration of physical, emotional, and spiritual aspects of health, acknowledging their profound interdependence in achieving overall well-being

In ancient holistic traditions like Ayurveda and traditional Chinese medicine, the concept of chakras, or energy centers, is foundational. It is believed that imbalances in these energy centers can manifest as physical or emotional ailments. Certain materials are thought to resonate with different chakras, each possessing unique vibrational frequencies capable of harmonizing and aligning these energy centers. This alignment is believed to facilitate the free flow of energy throughout the body, promoting overall wellness.

Furthermore, holistic practices often emphasize the significance of vibrational frequencies in influencing health. It is believed that everything, including our bodies and natural materials, emits vibrations at varying frequencies. Higher frequencies are associated with states of improved health and well-being, while lower frequencies may contribute to imbalance and discomfort.

By incorporating materials with higher vibrational frequencies individuals aim to elevate their energetic state and promote optimal health and vitality.

In essence, holistic wellness practices offer alternative pathways to well-being, emphasizing the interconnectedness of various dimensions of the self. By integrating certain natural materials into their surroundings, individuals tap into the innate healing power of nature, fostering harmony within themselves and their surroundings, while also aligning with higher frequencies conducive to holistic wellness.

In today’s society, individuals spend a significant portion of their lives sitting in chairs, sitting or lying on couches, lying in beds or in close proximity to any number of other types of furniture. From the corporate office to the home, people spend significant portions of their life interfacing and/or interacting with furniture. To this end, there is a need in the market to create furniture that can not only function as furniture but to also provide holistic and therapeutic health benefits to anyone using the furniture. There is a need in the market for furniture design to take advantage of the chakars, known healing frequencies and/or sacred geometries.

### SUMMARY OF THE EMBODIMENTS

Resonance frequency is the natural frequency at which an object vibrates or oscillates most easily. When a force is applied to an object at its resonance frequency, the amplitude of the vibrations increases significantly. This phenomenon is known as resonance, and it can occur in a variety of systems, such as mechanical, electrical, and acoustic systems.

The preferred embodiments of the products and methods disclosed herein use materials known to carry high energy frequencies and that are used in Ayurvedic and traditional Chinese medicine as a holistic way to support physical, emotional, and energetic wellbeing.

Incorporating frequencies into the furniture through carefully selected materials and placement of those materials can help to foster deeper grounding connection with nature and promote harmony between the mind, body and spirit.

The combination of copper and the powerful healing energy from crystals creates a grounding and calming experience, softening the energy of the day. The synergistic effects of these materials may amplify their health benefits, offering users a comprehensive wellness experience within their living spaces.

The furniture described in the embodiments herein consists essential of high frequency materials. Natural wood, is a core component and may be used for the structural members. Preferably, this wood is pine wood but other woods may be used. The furniture incorporates high-frequency metals, including a selection from copper, aluminum, brass, tin, iron, bronze, silver, gold, zinc, magnesium, titanium, and platinum. In other embodiments, the furniture incorporates high-frequency metals including a selection from copper, aluminum, silver, gold, brass, tin, zinc and iron.

The upholstery used in this furniture is crafted from high-frequency fabric. In most embodiments, between 14 and 22 yards of high frequency material may be used. Of course, other embodiments, such as an end table or other table may use way less and pieces such as sectional couches may use more. High-frequency fabric may be chosen from a variety of high-frequency materials, such as 100% Linen, or 100% wool. However, as explained in more detail herein, wool and linen should not be both used on the same piece of furniture, as it would result in zero frequency.

Inside the furniture, there are multiple high-frequency crystals placed in internal compartments. These crystals can range between one and ten pounds in total weight. For optimal performance, a plurality of copper balls is positioned in close proximity to the high-frequency crystals. The copper may amplify the effects of the crystals. In some embodiments, other forms of copper, such as copper mesh lined pockets, may be used to get the same amplification affect as copper balls.

The specific types of high-frequency crystals incorporated into the furniture include options such as red jasper, hematite, amethyst, rose quartz, clear quartz, citrine, yellow jasper, and rose quartz, either alone or in combination. Notably, some configurations feature amethyst and rose quartz crystals together. Additionally, the high-frequency crystals may be enclosed in copper mesh fabric pockets or may be foiled in copper.

This unique combination of materials and design elements culminate in a piece of furniture, such as a sofa, that harnesses the benefits of high-frequency components for enhanced functionality and aesthetic appeal.

In one embodiment, a sofa is provided. The sofa comprises a structural frame made of pine wood. The sofa uses high-frequency metals selected from the group consists of: copper, Silver, Gold, Zinc, Titanium, and Platinum. The sofa also uses upholstery and batting made from a high-frequency fabric selected from the group consisting of: cotton, silk, linen, wool, bamboo, hemp, and Tencel (Lyocell). In addition, the sofa comprises a plurality of high-frequency crystals located in internal compartments within the furniture wherein the high-frequency crystals are selected from the group consisting of: Red Jasper, Hematite, Amethyst, Rose Quartz, Clear Quartz, Citrine, Yellow Jasper and Rose Quartz. The sofa may also have a plurality of copper balls located in close proximity to the plurality of high frequency crystals.

A bed enclosure assembly is provided. In preferred embodiments, the bed enclosure assembly comprises a plurality of vertical walls spaced apart to form a plurality of openings coupled together by a ceiling and a floor. A plurality of doors are configured to close off the openings such that the enclosure can be completely closed off. A bed is positioned within the enclosure. The enclosure has copper cladding that covers each vertical wall in the plurality of vertical walls in its entirety. A plurality of crystals are located within the plurality of vertical walls of the bed enclosure 72.

### BRIEF DESCRIPTION OF THE DRAWINGS

FIG. 1 illustrates an embodiment of a holistic and therapeutic sofa designed using the principles taught herein;

FIG. 2 illustrates the location of different Chakras on the human body;

FIG. 3 illustrates an embodiment of a holistic and therapeutic sofa similar to the one in FIG. 1 but with an additional copper back;

FIG. 4A illustrates a top-down plan view of the sofa of FIGS. 1 and 3 with some exemplary dimensions for reference;

FIG. 4B illustrates a front elevation view of the sofa of FIGS. 1 and 3 with some exemplary dimensions for reference;

FIG. 4C illustrates a rear elevation view of the sofa of FIGS. 1 and 3 with some exemplary dimensions for reference;

FIG. 4D illustrates a left-side elevation view of the sofa of FIGS. 1 and 3 with some exemplary dimensions for reference;

FIG. 4F illustrates a right-side elevation view of the sofa of FIGS. 1 and 3 with some exemplary dimensions for reference;

FIG. 5A illustrates a top-down plan view of the interior of the sofa of FIGS. 1 and 3;

FIG. 5B illustrates a front elevation view of the interior of the sofa of FIGS. 1 and 3;

FIG. 6 illustrates an internal view of one embodiment of a sofa made according to the teachings herein; and

FIG. 7 illustrates an embodiment of a holistic and therapeutic bed designed using the principles taught herein.

FIG. 8 illustrates a different perspective view of the embodiment of a holistic and therapeutic bed shown in FIG. 7.

FIG. 9 illustrates a different perspective view of the embodiment of a holistic and therapeutic bed shown in FIGS. 7 and 8.

### DETAILED DESCRIPTION OF THE DRAWINGS

In preferred embodiments of the furniture disclosed herein, high-frequency materials are preferably used. High-frequency materials may aid in grounding, healing and overall wellbeing. In some embodiments, the furniture described herein differs from other furniture in that it consists essentially of only high-frequency materials. Accordingly, the materials used in the furniture is limited to high-frequency components and those that do not materially affect the basic and novel characteristics of the furniture. In such embodiments, only high-frequency materials are used for all the major components and fasteners are the only components that are not high-frequency components. In yet other embodiments, only high-frequency materials are used for all the major components and fasteners and filling/stuffing or batting for the cushions are the only components that are not high-frequency components. In still yet other embodiments, only high-frequency materials are used for all the major components including fasteners and only filling/stuffing/batting in the cushions is made from materials that are not high-frequency components. In still yet other embodiments, all of the components in the furniture, including the fasteners and stuffing/filling/batting are made from high-frequency materials. In such embodiments, the furniture is consisting of only high frequency materials.

There are a few metals that are considered to be high-frequency materials. One metal that is a high-frequency material is copper. Accordingly, in some embodiments, copper is used in the construction of the furniture. Copper is known to promote vitality and conductivity. Copper is renowned for its anti-inflammatory and antimicrobial properties. Studies have demonstrated that copper surfaces can effectively eliminate a wide range of pathogens, contributing to a cleaner, healthier environment. Copper is believed to resonate the body’s electromagnetic field, promoting vitality and energy flow. Copper is also a strong conductor and amplifier.

Copper may be used on the furniture in a decorative manner, structural component or simply added within the furniture for its healing powers or any combination thereof.

In addition to Copper, the following high-frequency metals are often referenced in holistic health practices for their potential healing effects as may be considered high-frequency materials as taught herein: Silver, Gold, Zinc, Magnesium Titanium, and Platinum. Each of these metals is believed to contribute uniquely to health and wellness through various mechanisms.

Silver is recognized for its antibacterial and antimicrobial properties. It has been utilized in various forms, including colloidal silver, believed to confer health benefits by inhibiting the growth of harmful microorganisms. This metal is often integrated into health products aimed at enhancing the body’s ability to resist infections.

Gold is associated with promoting healing and reducing inflammation, making it a valuable element in holistic health approaches. It is believed to balance energy within the body and is often employed in alternative therapies through topical applications and supplements designed to support overall wellness and vitality.

Zinc plays a critical role in immune function and wound healing. It is widely recognized in supplement form for its ability to sustain health and assist in cellular repair processes. This essential mineral is frequently included in health regimens aimed at bolstering the body's natural defenses.

As a vital mineral, magnesium is essential for numerous physiological functions and is widely used in supplements and topical formulations. It is particularly noted for its calming effects on the nervous system and its ability to relax muscles, thereby promoting overall physical well-being and stress relief.

Titanium is occasionally employed in therapeutic jewelry due to its lightweight properties and hypoallergenic nature. It is believed to contribute to energy balance in the body, making it beneficial in practices focused on promoting well-being and enhancing physical vitality.

Although less frequently referenced than other metals, platinum is known for its luxurious attributes and is thought to possess calming effects on the body. It may be utilized in holistic health applications to foster tranquility and emotional balance.

In addition to high-frequency metals, another high-frequency material that may be used are certain crystals. In some embodiments, crystals are used in the construction of the furniture. Amethyst and quartz are high-frequency crystals and believed to emit specific frequencies that promote energy balance and alignment within the body. Different crystals have different effects on different portions of the body and various examples of crystals being used in furniture are provided herein. Research suggests that certain crystals possess piezoelectric properties, meaning they can convert mechanical stress into electromagnetic energy, potentially aiding in harmonizing the body’s energy fields. Incorporating crystals into furniture design may help create an environment conducive to relaxation, stress reduction and overall well-being.

The choice of fabric within the furniture design is also an important aspect in preferred embodiments. Using high-frequency fabrics in furniture contributes to a more sustainable and healthy living environment, supports overall well-being, enhances comfort, and helps regulate indoor air quality. High-frequency fabrics also promote a sense of serenity through their breathable and hypoallergenic properties, making them suitable for various living spaces and personal preferences. Fabrics that are considered high-frequency include cotton, silk, linen, wool, bamboo, hemp, and Tencel (Lyocell). Furthermore, by choosing natural and sustainable materials for furniture, individuals can create a more harmonious home that fosters a connection to nature and supports eco-friendliness.

One of the most notable high-frequency fabrics is cotton. Renowned for its breathability and moisture absorbent qualities, cotton provides exceptional comfort, making it an ideal choice for furniture upholstery. Its natural hypoallergenic properties cater to sensitive individuals, and the fabric softens with every wash, enhancing the overall comfort and relaxation of any living space.

Silk is another luxurious fabric that adds an elegant touch to furniture design. Its smooth and soft texture not only evokes a sense of opulence but also contributes to a calming atmosphere. Silk naturally regulates temperature, ensuring comfort in various conditions, while its amino acids and proteins can also be beneficial for skin health, making it a unique choice for upholstered items.

Linen is well-loved for its durability and lightness. This fabric is highly breathable and effectively wicks moisture, making it perfect for warm climates. Linen's natural fibers possess antibacterial properties, promoting a healthier living environment. Its timeless aesthetic adds character and sophistication to any room’s decor.

In preferred embodiments, high-quality 100% black linen fabric is used. The selection of black linen is thoughtfully curated based on the principles of Chromology, allowing for a customized color experience that aligns with your unique preferences and the healing properties associated with each hue. Black linen may vibrate at 5,000 Hz.

The use of the materials to manufacture the sofa 10 discussed herein in conjunction with high-frequency linen to upholster the sofa 10 may create a calming, grounding environment that promotes relaxation, mental clarity, and emotional balance.

Wool is often sought after for its excellent insulating properties. Providing warmth without overheating, wool is a fantastic option for upholstery in colder climates. Additionally, its moisture-wicking abilities contribute to maintaining a dry and comfortable space. The natural elasticity of wool ensures that furniture retains its shape while providing necessary support.

The rise of eco-friendly options has brought bamboo to the forefront of sustainable furniture fabrics. Bamboo fibers are soft and breathable, making them an excellent choice for upholstery. The antimicrobial and hypoallergenic properties of bamboo enhance user comfort, while its sustainability roots promote an eco-conscious lifestyle.

Hemp fabric is increasing in popularity due to its remarkable durability and strength, which makes it resistant to wear and tear. This fabric also offers natural UV resistance, protecting both the fabric and the skin from sun exposure. With its breathable qualities, hemp contributes to comfort and longevity in indoor furnishings.

Lastly, Tencel (Lyocell) is a newer fabric that has gained recognition for its sustainability and luxurious feel. Made from sustainably sourced wood pulp, Tencel is highly breathable and moisture-wicking, helping maintain a fresh and comfortable environment. Its softness and gentle touch make it an excellent addition to any furniture, enhancing both style and comfort.

Certain fabrics can promote comfort and emotional wellness. The choice of fabrics can significantly impact emotional well-being and comfort levels. Natural fabrics such as cotton, linen, and wool are considered high-frequency materials and are preferred for their breathability and tactile properties and are believed to emit higher frequencies. Incorporating soft, natural fabrics into furniture design can enhance the overall sensory experience, promoting relaxation and emotional balance.

However, it has been found that when wool and linen are mixed together that their frequencies cancel each other out. Accordingly, wool and linen should not be used together in a single piece of furniture. Moreover, synthetic materials such as polyester and nylon are not high-frequency materials and resonate at 15 hz to 0 hz such that these fibers should never be used as they are destructive to the body. In contrast, both organic cotton, which resonates at 100 hz and cotton, which resonates at 70 hz,, are considered high-frequency materials.

Another high-frequency material is wood. Studies have shown that exposure to natural materials like wood can lower sympathetic nervous system activity, reducing stress levels and promoting relaxation. Furniture crafted from wood such as pine can enhance indoor air quality by regulating humidity levels and minimizing the presence of airborne toxins, fostering a healthier living environment.

Wood may be used in the construction of the furniture designs herein. In addition to wood’s high-frequency properties, wood offers both strength and aesthetic appeal. Various parts of furniture utilize wood in distinct ways to enhance functionality and design.

At the core of many furniture pieces is the frame. The frame may be made of wood to provide stability and support. Of course, the frame may also be made from a high-frequency metal. This structural framework is crucial for items like sofas, beds, and chairs, where durability is a must. Complementing the frame, legs play a significant role in the structure of furniture and may be crafted from wood or a high-frequency metal. The legs of tables, chairs, and cabinets not only support the weight of the furniture but also contribute to its overall visual appeal.

The tabletops of dining tables, coffee tables, and desks are often constructed from wood, providing a durable and attractive surface for everyday use. In addition, shelves made from wood are popular for bookcases, cabinets, and wall units, offering sturdy storage solutions that are both functional and visually pleasing. In other embodiments, the tabletops and shelves may also be construct from a high-frequency metal without departing from the teachings herein.

Seating furniture features backrests that may be made of wood or a high-frequency metal, adding style to chairs and benches. Moreover, arms on chairs and sofas may be crafted from wood or a high-frequency metal, further enhancing the aesthetic and providing support for users. When it comes to storage, drawer construction commonly incorporates wood, ensuring that drawers are both functional and long-lasting.

In cabinetry, cabinet doors are frequently made from wood, adding character and elegance to various storage solutions, including armoires and kitchen cabinets. To ensure structural integrity, supports and braces made of wood provide additional stability, especially in larger furniture items like tables and cabinets. In yet other embodiments, a high-frequency metal may be used to make the cabinet doors, supports or braces.

Bed frames may be made from wood, providing durability and a solid foundation for a good night’s sleep. In other embodiments, high-frequency metal may be used for the bed frame. In some embodiments, the bed frame is made from a blend of aluminum, copper, steel, and wood, ensuring durability and a solid foundation for restful sleep while effectively minimizing EMF exposure. Some designs may also incorporate high-frequency metals for enhanced performance.

In addition to structural elements, both wood and high-frequency metals may be utilized for decorative elements in furniture design. From moldings to inlays and intricate carvings, these details can significantly enhance the visual interest and unique character of each piece.

Through these various applications, it is clear that wood serves not only as a structural element but also as a key component in the aesthetic and functional qualities of furniture, making it a timeless choice for designers and craftsmen alike.

In preferred embodiments, one or more high-frequency materials such as copper, crystals, natural fabrics and wood are combined in a piece of furniture. One skilled in the art will appreciate from the teachings herein that any combination of copper, crystals, natural fabrics and wood may be used and, in some embodiments, one or more of those materials may be used in combination or individually. In addition, each material copper, crystals, natural fabrics and wood can be used in various different quantities depending on the size of the piece of furniture. As an example, copper and crystals may be used in one piece of furniture while copper, crystal and wood may be used in another. In yet another embodiment, all four of copper crystal, wood and natural fabrics may be used.

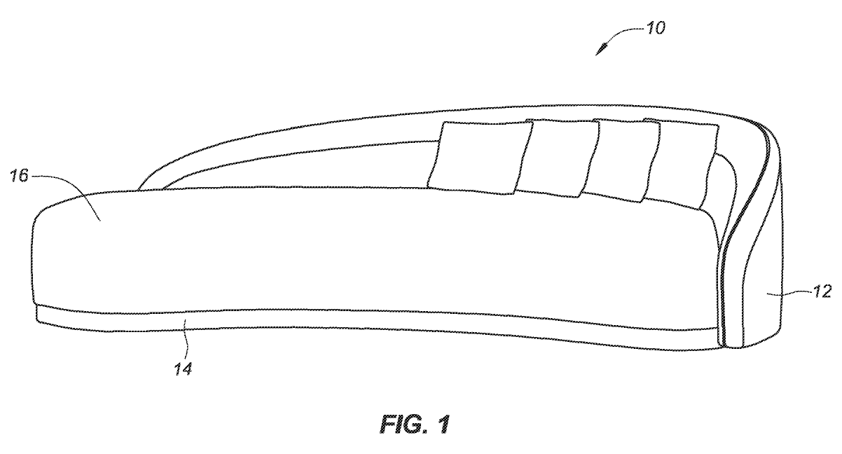

FIG. 1 illustrates an embodiment of a holistic and therapeutic sofa 10 designed using the principles taught herein. In preferred embodiments, sofa 10 includes 15 to 30 pounds of copper. As may be appreciated from FIG. 1, sofa 10 includes a copper back 12 and a copper toe kick 14. However, in other embodiments, sofa 10 may include copper in other locations and may or may not have a copper back 12 and/or copper toe kick 14.

In preferred embodiments, sofa 10 may include between 0.5 lbs. and 2 lbs. of copper balls (not shown in FIG. 1) located inside the sofa 10. In more preferred embodiments, sofa 10 includes 1 lb. of copper balls located inside the sofa 10. In preferred embodiments, the copper balls are located within the sofa 10 near where the heart and crown Chakra centers of the body would be located when a user is sitting or lying on the sofa 10. In yet other embodiments, the copper balls may be placed within the sofa 10 in other locations to align with other Chakra locations when a user is sitting or lying on the sofa 10.

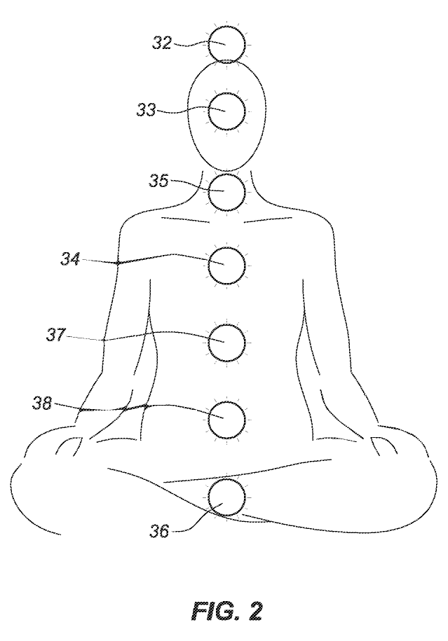

FIG. 2 illustrates the location of the various Chakra known in the human body. The present invention relates to the integration of crystals within furniture designs to enhance the alignment and balance of chakras. This innovative approach aims to incorporate the natural healing properties of crystals into everyday furniture, promoting wellness, tranquility, and positive energy in living spaces.

Looking at FIG. 2, the human body has seven primary chakras, which are energy centers that influence our physical, emotional, and spiritual well-being. The Root Chakra 36, located at the base of the spine, is associated with grounding and stability. The Sacral Chakra 38, situated in the lower abdomen, governs creativity and emotional connection. The Solar Plexus Chakra 37, located in the upper abdomen, is linked to personal power and self-esteem. The Heart Chakra 34, at the center of the chest, represents love and compassion. The Throat Chakra 35, found in the throat area, facilitates communication and self-expression. The Third Eye Chakra 33, located between the eyebrows, is associated with intuition and insight. Finally, the Crown Chakra 32, situated at the top of the head, connects us to higher consciousness and spiritual awareness.

The placement of crystals associated with each chakra within furniture aims to harmonize energy flow and improve the overall well-being of individuals using the furniture.

Some non-limiting examples of furniture designs incorporating the high-frequency materials are presented below. As one will appreciate, other designs for furniture may be created without departing from the scope of the teachings herein.

For the Base Chakra 36 (Root Chakra), linked with grounding and stability, high-frequency crystals can be strategically integrated into furniture made from high-frequency materials. For instance, storage ottomans crafted from sustainably sourced hardwood can feature hidden compartments with removable bottom panels that hold high-frequency crystals. In some embodiments, the compartments may be lined with natural cotton fabric to enhance energy flow. The use of natural dyes to color the fabric can further promote a harmonious atmosphere. Additionally, wooden bed frames made from reclaimed wood can be designed with hollow spaces in the legs, providing secure compartments for high frequency crystals, easily accessible through discreet openings underneath.

Although in various embodiments any high-frequency crystals may be used, in preferred embodiment, Red Jasper and Hematite may be used. Red Jasper and Hematite are particularly well known to be effective with the Base Chakra 36.

When addressing the Sacral Chakra 38, associated with creativity and emotional balance, furniture designs can benefit from multiple applications. A round dining table made from quality hardwood with a natural finish can incorporate an inner ring just beneath the tabletop to hold larger high-frequency crystal pieces. In preferred embodiments, the high-frequency crystals may be covered by a glass surface that allows for visibility.

In yet other embodiments, couches upholstered in organic linen fabric can enhance this energy by having snug compartments on the armrests for polished high frequency crystals, complete with cushioned wool liners and magnetic closures for easy access.

In targeting the Solar Plexus Chakra 37, which is linked to personal power and self-esteem, furniture such as coffee tables and bookshelves can be transformed to accommodate high-frequency crystals. In some embodiments, Citrine and Yellow Jasper may be used. In yet other embodiments, a coffee table constructed from a natural wood or high-frequency metal can feature a lower shelf equipped with a sliding drawer lined in natural wool to hold high-frequency crystals. Bookshelves made from natural wood can include illuminated sections behind frosted glass doors to display high-frequency crystals.

The Heart Chakra 34, associated with love and compassion, can be supported through furniture like accent chairs and bedside tables made from organic materials. Accent chairs crafted from sustainable timber can incorporate pockets that are designed to hold high-frequency crystals such as Rose Quartz pieces. Similarly, bedside tables made from natural wood or high-frequency metals can feature small drawers specifically for high-frequency crystals, allowing users to select different crystals based on their emotional needs each night.

To facilitate communication and self-expression associated with the Throat Chakram35, furniture designs for desks and bookshelves can include integrated crystal holders made of natural materials. A workstation made of polished hardwood may feature a recessed area within the desktop, lined with cotton velvet to securely hold high-frequency crystals such as Aquamarine or Blue Lace Agate crystals, complete with a decorative copper cover that doubles as a paperweight. Bookshelves constructed from natural wood can feature eye-level transparent compartments with glass fronts housing high-frequency crystals.

The Third Eye Chakra 33, connected to intuition and insight, can be supported through recliners and floor lamps made with high-frequency materials. Recliners made of sustainably harvested wood can be designed with cushioned headrests containing secure pockets for high-frequency crystals.

Floor lamps can feature bases made from natural wood with integrated holders, allowing light to glow through the crystals, illuminating the surrounding area and serving as a focal point for meditation.

The crown chakra 32 is believed to promote overall health and well-being on physical, emotional, mental, and spiritual levels. For the Crown Chakra 32, which connects to higher consciousness, furniture pieces such as sofas and recliners with headrests can be designed to incorporate high frequency crystals within the headrests. Sofas made from a framework of durable hardwood can include headrests with cushioned pockets specifically tailored for high-frequency crystals.

High vibrational crystals can be beneficial for grounding and blocking electromagnetic fields (EMFs). While no crystal can entirely block EMFs, certain stones are believed to help mitigate their effects and enhance your overall energy. A non-limiting list of crystals that may be used in the furniture described herein for grounding and EMF protection is provided.

Black Tourmaline has powerful protective qualities. Black tourmaline may absorb EMFs and negative energy. Black Tourmaline is a favorite for grounding and creating a protective shield around the user.

Hematite is a grounding stone that helps to absorb negative energy and can protect against EMFs. It is often used to enhance focus and concentration.

.Shungite is renowned for its ability to absorb and neutralize EMFs. It is composed of a unique form of carbon and is often used in water purification, as well as for protection against electromagnetic radiation. In the building of furniture, between the crystals Lemurian Quartz, Turmaline and Shungite, Shungite is the most important.

Selenite is not a grounding stone. However, Selenite has high vibrational energy that can help cleanse and charge other crystals. Selenite may create a protective energy field and can clear the environment of negative energy.

Smoky Quartz is a grounding stone that is effective at transmuting negative energy. It may also help protect against EMF radiation and promote emotional stability. d

Rose quartz is the stone of love and compassion. Rose quarty promotes emotional healing and self-love and is often used to create a calming atmosphere. While rose quartz is primarily recognized for its emotional and healing properties, some believe it can help mitigate negative energies, including those from EMFs. The gentle energy of rose quartz is thought to create a protective barrier against stressors in the environment. To use rose quartz for EMF protection, the rose quartz should be placed near the electronic devices, worn as jewelry, or kept in your living or working space to promote a peaceful environment.

Amethyst has calming and spiritual qualities. Amethyst may be used for meditation and spiritual growth, promoting clarity and a higher state of consciousness. Amethyst may protect against negative energies, including EMFs. Amethyst may create a protective shield that helps absorb and transmute harmful radiation. Amethyst can be placed in rooms where electronic devices are used most frequently, worn as jewelry, or used during meditation to help create a protective environment.

Using both rose quartz and amethyst together may enhance their protective properties. The combination may provide emotional support while also helping to counteract the potential negative effects of EMFs. There are various ways to combine rose quartz and amethyst to create their synergistic benefits. One method is to create a crystal grid by placing rose quartz and amethyst in patterns around your living space or work area to amplify their protective energies. Another method is to hold both crystals during meditation to enhance their effects on emotional healing and protection from EMFs.

Tiger's eye has grounding and protective properties. Tiger’s eye helps to enhance focus and clarity while providing a shield against negative influences, including EMFs.

Labradorite is a stone of transformation and protection. Labradorite may create a barrier against negative energies, including those from electronic devices.

Obsidian is a powerful grounding stone that can help absorb and repel negative energies. Obsidian is often used for protection and can aid in emotional healing.

Celestite has high vibrational energy and a connection to spiritual realms. Celestite may also help create a peaceful environment and mitigate stress from EMFs.

Black Kyanite is an effective earth and psychological healing tool. The striations rapidly move negativity out, aligning the Chakras. Black Kyanite draws stagnant energy from the physical body, replenishing it with the positive life-force.

Limestone is a sedimentary rock composed primarily of calcium carbonate, and while it is not typically highlighted specifically for its EMF blocking properties, it has some characteristics that can contribute to reducing electromagnetic exposure. Limestone is a natural material, which may create a grounding effect, promoting stability and reducing stress levels in a living environment. The density and structure of limestone may help in attenuating some forms of radiation, as denser materials may absorb certain frequencies better than lighter materials. However, the effectiveness of limestone specifically in blocking EMF radiation is not well-researched. Limestone often has a calming appearance and is sometimes used in holistic practices for its reported ability to create serene environments, which can contribute to overall well-being, including stress reduction from EMF exposure. Besides using limestone as a building material, incorporating limestone sculptures or decor into a space may help create a balanced energy, contributing to a peaceful atmosphere.

While limestone, or any other crystal or stone discussed herein, in isolation has some appealing qualities and may contribute to a calming environment, it is essential to approach EMF protection with a combination of materials and practices for the best outcomes. Always consider scientific research and practical solutions alongside natural approaches for managing EMF exposure.

Within any piece of furniture, especially sofas, loveseats and recliners, compartments or pockets can be made to hold copper balls, crystals or other high-frequency enhancing materials. One skilled in the art of furniture making, will appreciate where within sofa 10 the copper balls could be placed to align with the crown Chakra 32 and the heart Chakra 34 or any other Chakra. In particular, copper is known to resonate with the root Chakra 36 and sacral Chakra 38.

Copper is believed to enhance the energy conductivity of crystals, allowing for a stronger flow of energy. The combination of copper and crystals creates a synergistic effect, magnifying the benefits and promoting balance and well-being. By incorporating copper into holistic wellness practices, individuals can align their Chakras, amplify energy flow, and enhance the benefits of crystals, ultimately promoting harmony and well-being in body, mind, and spirit.

In preferred embodiments, sofa 10 includes between 1 and 50 lbs of crystals. In even more preferred embodiments, the sofa contains between 25 and 30 lbs of crystals. In still yet other embodiments, the sofa contains between 1 and 10 lbs of crystals (not shown in FIG. 1). Crystals aid in balancing all the chakras. In yet more preferred embodiments, sofa 10 may include 3 lbs. to 4 lbs. of crystals. In some embodiments, the sofa 10 may include 3 lbs. to 4 lbs. of amethyst and rose quartz crystals in combination. Amethyst and rose quartz crystals vibrate at 852 hz. In yet other embodiments, the sofa 10 may include 3 lbs. to 4 lbs. of clear quartz crystals which vibrate at 963 hz. In some embodiments, clear quartz may be used. In some embodiments, Lemurian clear quartz may be used. In one preferred embodiment, 29 lbs. of Lemurian clear quartz are used.

Lemurian clear quartz is a unique variety of quartz known for its distinctive striations or "barcodes" on one side of the crystal, which are said to contain the record of ancient wisdom and consciousness from the lost civilization of Lemuria. This crystal is revered for its healing properties, amplifying energy, and ability to promote spiritual growth. Lemurian clear quartz can be a valuable tool for supporting EMF protection and enhancing overall energetic well-being. While it may not block EMFs entirely, its amplifying, cleansing, and healing properties can help create a more harmonious environment in the presence of electromagnetic fields. For optimal EMF protection, consider using Lemurian clear quartz in conjunction with other protective practices, such as reducing exposure to electronic devices and fostering a low-EMF living space.

Lemurian clear quartz may be used by positioning Lemurian clear quartz in rooms where electronic devices are frequently used, such as home offices or bedrooms, to help absorb and mitigate EMFs. Incorporating Lemurian clear quartz into a crystal grid with other protective stones (such as black tourmaline or selenite) may amplify their protective and cleansing properties.

In preferred embodiments, sofa 10 has Elite Shungite crystals strategically integrated into its design to neutralize electromagnetic fields (EMF) and boost the overall well-being of anyone using the sofa 10. In some embodiments, the sofa 10 has 29 lbs. of clear quartz and Shungite in combination.

In preferred embodiments, the sofa 10 is crafted to shield a user from harmful electromagnetic fields. With the sofa’s unique combination of Clear Quartz, Shungite, and copper, the sofa 10 is equipped with powerful protective properties, allowing a user to relax without the worry of excessive EMF exposure.

Clear quartz is known as a "master healer" and is believed to amplify energy and intention. This property can be particularly useful when combined with other healing stones or practices aimed at EMF protection, such as Elite Shungite. The combination of shungite and clear quartz “the Master Healer” in furniture is thought to provide significant protection against EMFs, with Shungite actively absorbing and neutralizing harmful frequencies while clear quartz enhances the protective energy, while keeping the Shungite charged. The combination of Lemurian Clear Quartz and Shunghite enhances grounding abilities and neutralizes EMFs.

In yet other embodiments, Citrine (“Sun Stone”) may be used as the high-frequency crystal.

The crystals may be amplified by the pure copper balls. The copper amplification can double the energy of the crystals.

In preferred embodiments, a high frequency material 16 is used for the upholstery of the sofa 10. In preferred embodiments, the high frequency material 16 is wool. Pure wool resonates at a tremendous 5000 hz – (50 times the natural frequency of the human body!). Thus, the healing and positive potential of wool on the body is exponentially influencing our energy and health. In the embodiment shown in FIG. 1, 54 yards of wool upholstery is used. In other embodiments, more or less wool may be used depending on the size of the piece of furniture.

In some embodiments, copper fabric may be included. Copper fabric can amplify the wool frequency of 5000hz to increase exposure of the body to even higher frequencies. In preferred embodiments, between 1 and 20 yards of copper fabric are used. In more preferred embodiments, between 5 and 15 yards of copper fabric are used. In yet even more preferred embodiments, between 9 and 11 yards of copper fabric are used. In the embodiment shown in FIG. 1 10 yards of coper fabric is used.

The sofa 10 shown in FIG. 1 exposes the user to 963 hz + 852 hz + 5000 hz = 6,815 hz plus exposure to unique frequencies influencing our energy and health to give over 100 hz exposure to high frequency.

As mentioned above, wood is preferably used in the sofa 10. In preferred embodiments, Pine is used as the wood. Pine is the tree of Peace, and a Symbol of Purification & Cleansing. Pine contains chemicals that might have activity against pain and swelling (inflammation). It also seems to be mildly effective in killing bacteria and fungus.

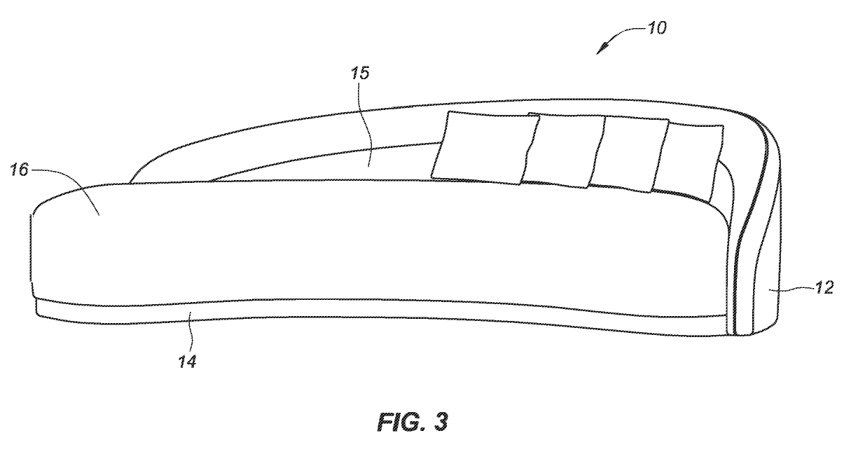

FIG. 3 illustrates an embodiment of a holistic and therapeutic sofa similar to the one in FIG. 1 but with an additional copper back 15.

In preferred embodiments, the furniture, like the sofa shown in FIGS. 1 and 3 for example, consists of: 1.) Pine wood frames; 2.) copper fabric backing pockets with foiled gemstones; and 3.) softened edges consistent with sacred geometry. In some embodiments, the furniture may also include copper mesh fabric pockets to be filled with gemstones/crystals. In preferred embodiments, the gemstones/crystals may be foiled in copper to amplify the frequency flow into the body, enhancing energy flow. In some embodiments, the clients may select the crystals/gemstones to be filed from the group consisting of amethyst, Citrine, clear quartz and rose quartz.

In some embodiments, the crystals are interconnected for enhanced amplification, secured with copper wire, and housed within a pure copper Faraday fabric pouch in the crystal compartment or pocket for additional effectiveness. Furthermore, in some embodiments, the Copper pouch is infused with lavender, providing a therapeutic scent that supports optimal healing and relaxation. Other scents may be used in other embodiments and the scents may be infused in other areas of the sofa 10.

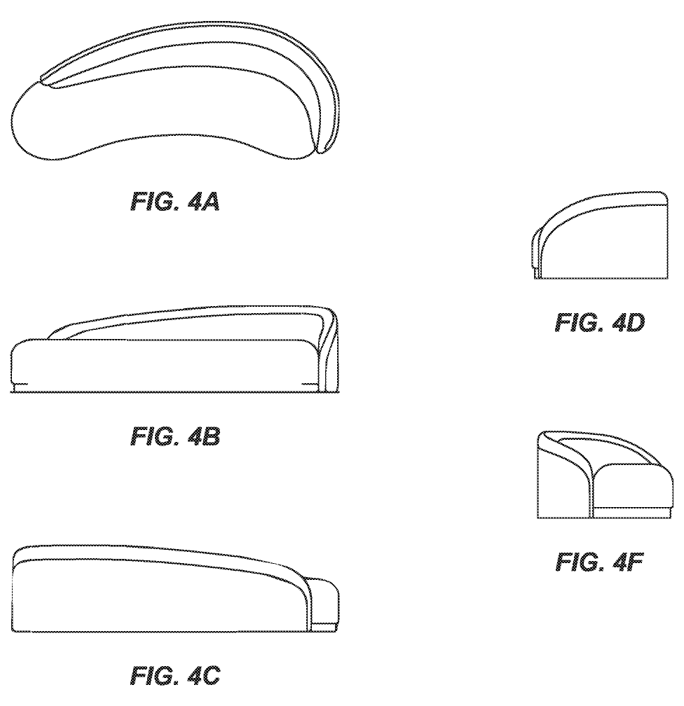

FIGS. 4A-4F illustrate various views of the sofa of FIGS. 1 and 3 with some dimensions for reference. As one skilled in the art will appreciate, these dimensions are just an example of one embodiment and other embodiments may have different dimensions.

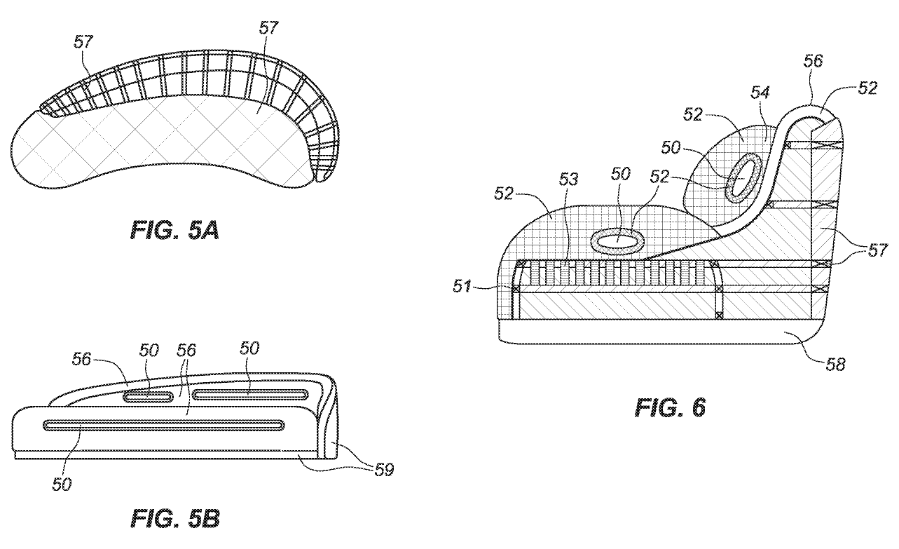

FIG. 5A-5C illustrates various interior views of the sofa of FIGS. 1, 3 and 4. FIG. 6 illustrates an internal view of one embodiment of a sofa made according to the teachings herein.

As may be seen in FIGS. 5A-5C and 6, crystals may be inserted inside inserts 50 with silk envelopes wrapped in copper mesh fabric. As may be seen, the crystals 50 may extend down the longitudinal length of the sofa 10 in various different locations. The crystals and pure copper balls wrapped in copper mesh fabric envelopes encased in silk. The crystals may be in both the back portion of the seat portion of the sofa or both.

In the embodiment of a sofa shown in FIGS. 5A-5C, the sofa is made with a certified sustainable green pine wood frame 57 and has a copper mesh fabric backing 51. The pine wood frame 57 included both horizontal and vertical members.

The sofa is filled with a sustainable batting 52 in numerous locations. In preferred embodiments, the batting is made from a high-frequency natural material like wool but in other embodiments, the batting made from a material that is non-high-frequency. In preferred embodiments, the batting 52 has a copper mesh fabric.

The sofa has spring metal coils 53. Although preferably made from a high frequency metal, the spring metal coils 53 may be made from a non-high-frequency metal without departing from the scope of furniture consisting essential of high-frequency materials as taught herein.

The upholstery fabric 56 is customizable and has a copper mesh fabric backing.

The sofa features inserts with crystals and pure copper balls 50 wrapped in copper mesh fabric envelopes encased in silk. The sofa has decorative zippers 54 that allow you to change the crystal inserts 50 inside.

The sofa has a certified sustainable green pine wood base 58 wrapped with 22 gauge copper. The copper 59 may be used on the back and base in a decorative way.

One aspect of the inventions taught herein lies as much in the absence of certain materials as much as it does in their presence. For example, the present application contemplates furniture made entirely of high frequency materials such as high frequency materials with the absence of low frequency materials where higher frequency materials exist. For example, furniture may be made of the materials discussed herein and no other materials or at least not other materials of any significant quantity. Accordingly, in some embodiments, the sofa 10 is constructed entirely of pine, wool, copper and crystals.

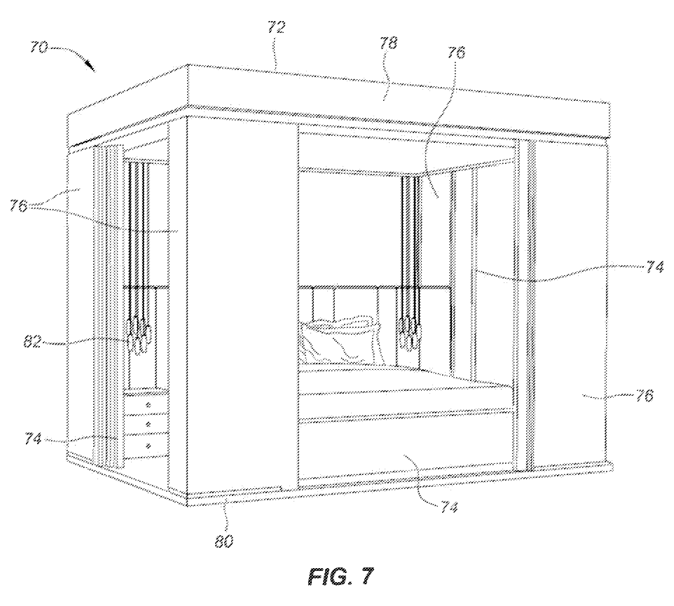

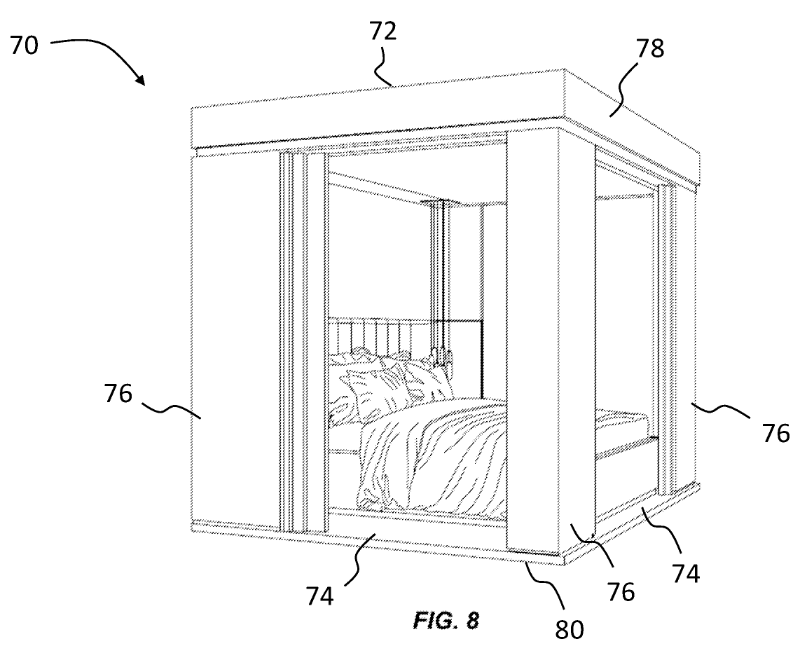

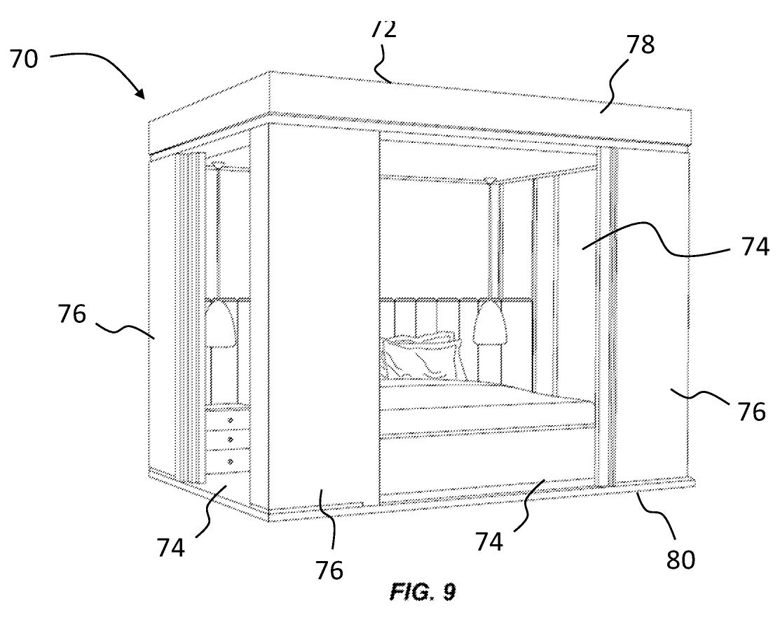

FIG. 7 illustrates an embodiment of a holistic and therapeutic bed designed using the principles taught herein. In preferred embodiments, the bed itself is inside an enclosure 72 that has openings 74. The openings are formed by the vertical walls 76 of the enclosure 72, which are coupled to the ceiling 78 and spaced apart to form the openings 74.

In preferred embodiment, the enclosure 72 has a floor 80 such that the bed itself is completely enclosed by the enclosure 72. In other embodiments, no floor 80 is used and the enclosure sits directly on the floor of the home.

Depending on the embodiment, the enclosure 72 may have any number of openings. At least one is required to allow ingress and egress of the enclosure. In preferred embodiments, the enclosure 72 has three openings, one each side of the bed and at the end but not in the back. However, other embodiments may have two or four openings.

In the embodiment shown in FIG. 7, the vertical walls 76 have an L-shaped cross-section and are located on the corners of the enclosure 72. However, in other embodiments, the vertical walls 76 may have other cross sections, such as rectangular for example.

In some embodiments, each vertical structure may be identically made and assembled on each corner to reduce costs. In other embodiments, different shaped and/or sized vertical walls 76 may be used.

In some embodiments, the enclosure 72 has an opening in the back wall behind the bed. However, in other embodiments, the back wall is closed off and a continuous wall from the floor 80 to the ceiling 78.

In preferred embodiments, the openings 74 may be closed off by doors. In preferred embodiments, these doors may be pocket doors that retract back into the vertical walls 76. In other embodiments, the doors may be accordion style doors other types of doors as those skilled in the art would appreciate. However, using pocket doors creates a sleeker design when the doors are open. Ideally, the enclosure 72 may be completely closed off using the plurality of doors. To this end, in preferred embodiments, each opening has a door that can close off the opening completely.

In some embodiments, the doors may be operated manually. However, in preferred embodiments the doors are motorized, for example with an electric motor. Ideally, the electric motor or motors is/are hidden in the ceiling 78 or vertical walls 76 so that they are concealed from sight and can also be sound dampened. The advantage of motorized doors is that the occupant/user of the bed 70 can control the doors without getting out of bed. This may be especially important when waking up in the morning and wanting access to light to help the natural wake up process.

In preferred embodiments, the enclosure 72 is manufactured from a EMF shielding metal. In some embodiments, the enclosure 72 is manufactured using traditional manufacturing methods and then clad in an EMF shielding metal. Ideally, the EMF shielding metal is copper but other metals that shield EMF may be used. If the enclosure 72 is clad in a EMF shielding metal, almost all the surfaces are clad. For example, the vertical walls 76 may be clad in their entireties. By “clad in their entireties” it is meant that no meaningful amount of surface area remains exposed and unclad. In some embodiments, one or both of the ceiling and the floor are also either made of copper or clad in copper.

In other embodiments, other metal combinations may be used for the enclosure to optimize EMF shielding. When considering the best metal combination for ultimate EMF protection, it's important to consider the specific types of EMF you want to shield against (e.g., low frequency, radio frequency, etc.), as well as the environment in which the shielding will be applied (e.g., residential, industrial).

For effective EMF protection in a building enclosed bed structure, various metals can be utilized to achieve comprehensive electromagnetic shielding across a broad frequency spectrum. Copper and aluminum are particularly suitable for high-frequency EMF attenuation, with copper offering excellent conductivity that effectively reduces RF signals and is commonly integrated into shielding fabrics and enclosures. Aluminum, being lightweight and cost-effective, provides effective shielding against both radio frequency and microwave frequencies, and can be used in combination with other materials to enhance overall performance. Steel, especially ferromagnetic steel, is effective for shielding low-frequency magnetic fields and offers substantial protection against electromagnetic interference from electrical devices. When paired with copper, this combination enhances shielding effectiveness across multiple frequency ranges. Mu-metal, a nickel-iron alloy specifically designed for magnetic shielding, effectively attenuates low-frequency magnetic fields and is ideal for sensitive electronic equipment; combining mu-metal with copper allows for broad-spectrum EMF shielding that covers both low and high-frequency ranges. Additionally, silver, known for its superior electrical conductivity, provides excellent high-frequency EMF protection; although more expensive and less common than copper, pairing silver with copper can offer an optimal balance of performance and cost-efficiency for EMF shielding in the intended structure. Beryllium Copper may also be used in EMF shielding.

In embodiments of a bed enclosure that provides effective EMF shielding, the utilization of graphene, in combination with metals such as copper or aluminum, offers notable advantages due to its exceptional electrical and thermal properties. Graphene, a single layer of carbon atoms arranged in a two-dimensional lattice, possesses high electrical conductivity, enabling it to efficiently reflect and absorb electromagnetic waves across a broad spectrum of frequencies. Its lightweight nature makes it an attractive material for applications where reducing overall weight is desirable, such as in portable or aerospace environments. Furthermore, graphene’s inherent flexibility allows it to be integrated into various substrates, facilitating the development of conformable and versatile shielding structures. Its ultra-thin profile enables effective electromagnetic interference (EMI) shielding with minimal bulk, making it especially suitable for space-constrained applications. When combined with metals like copper or aluminum, graphene can enhance the overall shielding effectiveness by leveraging their complementary properties, thereby creating a layered or composite shielding system capable of attenuating a wide range of EMF frequencies with improved efficiency and reduced weight.

In bed enclosure embodiments that use Graphene, the Graphene may be incorporated into the bed enclosure in numerous different ways to provide EMF shielding. In some embodiments, Graphene may be used in composite materials by integrating graphene into polymer matrices, creating layered or laminated shielding panels that can be incorporated into the bed’s structure. These composites can provide enhanced EMI filtering, protecting occupants from electromagnetic interference from external sources such as wireless signals and electrical devices commonly present in modern environments. Additionally, graphene can be embedded into textiles used within the bed enclosure, forming smart fabrics that serve as flexible, lightweight shielding layers for mattress covers or interior linings. These textiles can be designed not only to attenuate EMF exposure but also to incorporate sensing functionalities, such as detecting temperature, humidity, or even electromagnetic flux levels, thereby enabling real-time monitoring of the environment within the bed. Furthermore, graphene-based conductive coatings can be applied to various surfaces of the enclosure—such as interior panels, bed frame components, or decorative elements—to create a continuous and uniform EMI shield. These coatings can be formulated to maintain the structural integrity of the enclosure while providing effective electromagnetic attenuation against a broad spectrum of radiofrequency and low-frequency magnetic fields. Collectively, these graphene applications contribute to a comprehensive EMF shielding solution that enhances occupant safety and comfort within the enclosed bed environment.

Concrete, and in particular concrete blocks, may also provide effective EMF shielding and may be used in portions of the bed enclosure. For example, enclosure walls or portions thereof could be made from concrete or concrete blocks. When concrete blocks are used, these blocks typically consist of standard concrete mixed with materials that enhance electromagnetic shielding, such as ferromagnetic materials or conductive additives.

When using materials to provide EMF shielding, the materials may be used in numerous ways. The metals or concrete may be used in the structure of the enclosure. Sensitive components or sources of EMF that are part of the enclosure may be wrapped or enclosed to prevent transmission to other parts of the enclosure. Panelling or coverings may be constructed out of EMF material and then applied to the inside, outside or both of the enclosure. Linens may be woven with EMF shielding materials such that additional protection is provided to a user. In addition, metals such as aluminium and copper that have high conductivity properties may be used for grounding the system or systems. Effective grounding can help mitigate EMI issues by providing a low-resistance path for unwanted electrical currents.

Some embodiments of the bed 70 include red lights. In preferred embodiments, the red lights are located on the ceiling 78 and located and oriented such that they can provide red light therapy to an occupant or user of the bed. In yet other embodiments, the bed 70 may have red lights for red light therapy in other locations in addition to the ceiling. Such locations can be on the inside of the vertical walls for example.

The embodiments that include red lights permit the users to benefit from red light therapy (RLT). RLT is a treatment that utilizes low-level wavelengths of red light to stimulate various biological processes within the body. In preferred embodiments, RLT uses near-infrared light (NIR). Over recent years, RLT has gained significant popularity for its potential to offer a wide range of health benefits. One of RLTs most common applications is in skin health. RLT is frequently used to improve conditions such as acne, psoriasis, and eczema. RLT may also help reduce the appearance of wrinkles, fine lines, and age spots by encouraging collagen production and supporting skin regeneration.

Another notable benefit of RLT is its ability to accelerate wound healing. By enhancing cellular energy production and reducing inflammation, RLT supports tissue repair and can promote faster recovery from cuts, wounds, and injuries. RLT also shows promise in providing pain relief. Conditions like arthritis, joint pain, and muscle soreness may be alleviated through the anti-inflammatory and healing effects of RLT, offering a noninvasive alternative for pain management.

Athletes and fitness enthusiasts often turn to RLT to enhance muscle recovery following strenuous exercise. The treatment can reduce muscle soreness and improve recovery times by increasing blood circulation and decreasing inflammation. RLT has also been used to support hair growth, especially in individuals with conditions like androgenetic alopecia (commonly known as male or female pattern baldness). By stimulating the hair follicles, it may help encourage natural hair regrowth.

Beyond physical recovery, RLT may influence mental and emotional well-being. Some studies suggest that exposure to red light can help regulate circadian rhythms, potentially improving sleep quality. In addition, RLT has been associated with mood enhancement, possibly due to its effects on brain chemistry and hormonal balance. RLTs anti-inflammatory properties make it beneficial for a variety of conditions, including chronic pain, autoimmune disorders, and certain skin diseases.

Emerging research also indicates that RLT may support metabolic function, aiding in weight management and fat loss by enhancing mitochondrial activity. Some findings suggest benefits for dental health as well, including reduced gum inflammation and improved healing post-dental procedures. Additionally, RLT may boost immune function, contributing to overall health and resilience against illness.

In the realm of cognitive health, early studies show that RLT could have neuroprotective effects, potentially improving cognitive function and reducing the risk of neurodegenerative diseases. While many individuals report positive outcomes from red light therapy, it's important to approach the treatment with realistic expectations. The effectiveness of RLT can vary depending on the specific condition being treated, the consistency of use, and individual biological differences.

Some embodiments of the bed 70 include speakers. In preferred embodiments, the speakers are built into the vertical wall 76 or ceiling 78 or both. In preferred embodiments, the speakers are located and oriented such that they can provide sound therapy to an occupant or user of the bed. In yet other embodiments, the bed 70 may have sound for sound therapy in other locations such as below the bed or behind the bed. These speakers may be in addition to or instead of the speakers in the vertical walls 76 and/or ceiling 78.

In some embodiments, the speakers are used to play Tibetan bowl sound healing. In preferred embodiments, the speakers have adjustable frequency levels customized to a user’s unique healing needs. This one-of-a-kind auditory experience promotes relaxation and deepens a user’s connection to holistic wellness.

In preferred embodiments, the bed 70 further includes a CPU or other controller such as a micro-controller or the like and interface to allow scheduling of the various elements. For example, a user may desire the motorized doors to open at a certain time in the morning to allow nature light in to wake up the user more naturally. The speakers may also come on automatically at a scheduled time to further assist the wake-up process. The red lights or other lighting may also come on at a particular time and some of the lights may be on dimmers such that they brighten gradually with time. Shielding will be important to ensure any incorporated electronics do not radiate EMF into the interior of the bed 70.

In preferred embodiments, the bed 70 may include gem stone chandeliers. One or more gem stone chandeliers may be placed in and around the sleeping area.

As discussed above, the positive vibrational effects of various crystals may be amplified by copper. Accordingly, in some embodiments, crystals are built-in to the enclosure such that they are amplified by the copper. In preferred embodiments, the crystals may be embedded into the vertical walls 76 and amplified by the copper in the vertical walls 76.

Some embodiments of the bed include additional cabinetry compartments designed to house the crystals, allowing for easy charging and convenient removal or replacement based on your preferences.

Similar to the saft discussed herein, the mattress may have crystals within the mattress. As also explained herein, low frequency fabrics may be used. In preferred embodiments, 100% Mulberry silk bedding may be used in combination.

In some embodiments, the actual sleeping bed and/or bedding may be provided separately from the enclosure 72. However, preferably, the sleeping bed and bedding are provided in combination with the enclosure 72.

The embodiments herein are designed to allow the user to benefit from whole body vibration (WBV). WBV involves standing, sitting, or lying on a machine equipped with a vibrating platform or designed to vibrate the body. These vibrations stimulate the muscles to contract and relax, mimicking the effects of physical activity and offering a range of potential benefits. One of the primary advantages of WBV is the enhancement of muscle strength and tone, particularly in the lower body, as the repeated muscle contractions contribute to improved muscular development. Additionally, WBV can aid in increasing flexibility by promoting muscle relaxation and expanding the range of motion in the joints.

Research also suggests that WBV may help increase bone mineral density, making it a potentially valuable option for individuals at risk of osteoporosis or other bone-related conditions. Another key benefit is improved blood circulation; the vibrations stimulate better blood flow, enhancing the delivery of oxygen and nutrients throughout the body. Regular use of WBV has been linked to better balance and coordination, which is particularly beneficial for older adults or individuals undergoing rehabilitation.

When combined with a healthy diet and exercise routine, WBV may support weight loss and improved body composition, though outcomes can vary from person to person. It may also assist in reducing muscle soreness and shortening recovery time after workouts by improving circulation and promoting muscle relaxation. Furthermore, WBV can stimulate the lymphatic system, which supports detoxification and boosts immune function.

Some individuals report pain relief from conditions such as arthritis or back pain, though these effects can be highly individual. WBV, like other forms of exercise, is known to trigger the release of endorphins, which can enhance mood and alleviate feelings of stress and anxiety. Another notable aspect of WBV is its accessibility; because it can be performed in a low-impact manner, it may be suitable for individuals with limited mobility or those recovering from injuries. The effectiveness of WBV may vary based on individual factors, including the frequency and duration of use.

What is claimed is:

1. A bed enclosure assembly comprising: a plurality of vertical walls spaced apart to form a plurality of openings; a ceiling coupled to a top of each vertical wall in the plurality of vertical walls; a floor coupled to a bottom of each vertical wall in the plurality of vertical walls; a plurality of doors wherein each door in the plurality of doors is configured to close off at least one opening in the plurality of openings; a bed, positioned within the enclosure; copper cladding that covers each vertical wall in the plurality of vertical walls in its entirety; and a plurality of crystals located within the plurality of vertical walls.

2. The bed enclosure assembly of claim 1, further comprising red lights configured for red light therapy use.

3. The bed enclosure assembly of claim 2, further comprising a plurality of speakers within the bed enclosure assembly.

4. The bed enclosure of claim 1, wherein each vertical wall in the plurality of vertical walls is located on a corner.

5. The bed enclosure of claim 4, wherein each door in the plurality of doors is a pocket door that may be stowed in the plurality of vertical walls.

6. The bed enclosure of claim 1, wherein the plurality of crystals are selected from the group consisting of Red Jasper, Hematite, Amethyst, Rose Quartz, Clear Quartz, Citrine,Yellow Jasper and Rose Quartz.

7. The bed enclosure assembly of claim 1, wherein the plurality of crystals are high frequency crystals.

8. The bed enclosure assembly of claim 7, wherein a plurality of copper balls is located in close proximity to the plurality of high frequency crystals.

9. The bed enclosure assembly of claim 7, wherein the plurality of high-frequency crystals are enclosed in copper mesh fabric pockets.

10. The bed enclosure assembly of claim 7, wherein the plurality of high frequency crystals are amethyst and rose quartz crystals in combination.

11. The bed enclosure assembly of claim 7, wherein the plurality of high-frequency crystals are foiled in copper.

12. A bed enclosure assembly comprising: a plurality of vertical walls spaced apart to form a plurality of openings; a ceiling coupled to a top of each vertical wall in the plurality of vertical walls; a floor coupled to a bottom of each vertical wall in the plurality of vertical walls; a plurality of doors wherein each door in the plurality of doors is configured to close off at least one opening in the plurality of openings; a bed, positioned within the enclosure; EMF cladding that covers each vertical wall in the plurality of vertical walls in its entirety; a plurality of red lights coupled to the vertical walls or ceiling and configured for red light therapy use; and a plurality of high frequency crystals located within the plurality of vertical walls.

13. The bed enclosure assembly of claim 12, wherein the EMF cladding is copper.

14. The bed enclosure assembly of claim 12, further comprising a plurality of speakers within the bed enclosure assembly.

15. The bed enclosure of claim 12, wherein each vertical wall in the plurality of vertical walls is located on a corner.

16. The bed enclosure of claim 12, wherein the plurality of high frequency crystals are selected from the group consisting of Red Jasper, Hematite, Amethyst, Rose Quartz, Clear Quartz, Citrine, Yellow Jasper and Rose Quartz.

17. The bed enclosure assembly of claim 12, wherein a plurality of copper balls is located in close proximity to the plurality of high frequency crystals.

18. The bed enclosure assembly of claim 12, wherein the high-frequency crystals are enclosed in copper mesh fabric pockets.

19. The bed enclosure assembly of claim 12, wherein the high frequency crystals are amethyst and rose quartz crystals in combination.

20. The bed enclosure assembly of claim 12, wherein the high-frequency crystals are foiled in copper.
